(19) United States
(12) Patent Application Publication
(10) Pub. No.: US 2024/0148682 A1
(43) Pub. Date: May 9, 2024

(54) METHOD FOR REGULATING DOWNSTREAM SIGNALING PATHWAY MEDIATED BY ACTIVATION OF TRIMETHYLAMINE-N-OXIDE

(71) Applicant: China Medical University, Taichung City (TW)

(72) Inventors: Feng-Yao Tang, Taichung City (TW); En-Pei Isabel Chiang, Taichung City (TW)

(73) Assignee: China Medical University, Taichung City (TW)

(21) Appl. No.: 18/347,217

(22) Filed: Jul. 5, 2023

(30) Foreign Application Priority Data
Nov. 9, 2022 (TW) 111142768

(51) Int. Cl. A61K 31/202; A61P 29/00; A61P 31/14; A61P 39/06

(52) U.S. Cl. CPC A61K 31/202; A61P 29/00; A61P 31/14; A61P 39/06

(57) ABSTRACT

A method for regulating a downstream signaling pathway mediated by an activation of a trimethylamine-N-oxide includes administrating an omega-3 polyunsaturated fatty acid to a subject in need thereof. The omega-3 polyunsaturated fatty acid is for inhibiting an inflammatory response, for inhibiting an infection of a SARS-CoV-2 to a cell, for reducing an oxidative stress level in the cell, for alleviating an impairment of neovascularization, or for inhibiting a vascular fibrogenesis.

### RELATED APPLICATIONS

This application claims priority to Taiwan Application Serial Number 111142768, filed Nov. 9, 2022, which is herein incorporated by reference.

### BACKGROUND

### Technical Field

The present disclosure relates to a method for regulating a downstream signaling pathway mediated by an activation of a trimethylamine-N-oxide. More particularly, the present disclosure relates to a method for regulating the downstream signaling pathway mediated by the activation of the trimethylamine-N-oxide by administrating an omega-3 polyunsaturated fatty acid.

### Description of Related Art

Trimethylamine-N-oxide (TMAO) is a metabolite generated by human intestinal bacteria. According to the current research, trimethylamine-N-oxide is mainly derived from incompletely digested carnitine and choline in the diet. Trimethylamine-N-oxide is able to enter the blood circulation and promotes the accumulation of macrophages on the blood vessel wall, inhibits intestinal cholesterol absorption, and induces platelet hyperactivity, thereby increasing the risk of atherosclerosis and thrombosis.

Further, trimethylamine-N-oxide is able to stimulate endothelial cells of a blood vessel to transform into myofibroblasts and promotes vascular fibrogenesis, wherein the vascular fibrogenesis hardens the skin and induces degeneration of internal organs.

As mentioned above, researchers in academia and industry are actively developing methods to reduce the concentration of trimethylamine-N-oxide in vivo or to inhibit the activity of trimethylamine-N-oxide so as to improve or regulate the symptoms and signs associated with the activation of trimethylamine-N-oxide.

### SUMMARY

According to one aspect of the present disclosure, a method for regulating a downstream signaling pathway mediated by an activation of a trimethylamine-N-oxide includes administering an effective amount of an omega-3 polyunsaturated fatty acid to a subject in need thereof.

### BRIEF DESCRIPTION OF THE DRAWINGS

The present disclosure can be more fully understood by reading the following detailed description of the embodiment, with reference made to the accompanying drawings as follows:

FIG. 1 shows the results of the Western blot analysis of the inhibition of the omega-3 polyunsaturated fatty acids to the factors related to the downstream signaling pathways mediated by the activation of the trimethylamine-N-oxide.

FIG. 2 shows the results of the Western blot analysis of the inhibition of the omega-3 polyunsaturated fatty acids to other factors related to the downstream signaling pathways mediated by the activation of the trimethylamine-N-oxide.

FIG. 3 shows the results of the inhibition of the omega-3 polyunsaturated fatty acids to the expression of miR-221 mediated by the activation of the trimethylamine-N-oxide.

FIG. 4 shows the results of the inhibition of the omega-3 polyunsaturated fatty acids to the expression of IL-6 mediated by the activation of the trimethylamine-N-oxide.

FIG. 5 shows the results of the inhibition of the omega-3 polyunsaturated fatty acids to the trimethylamine-N-oxide activation-mediated entry of SARS-CoV-2 plasmid into human endothelial progenitor cells.

FIG. 6 shows the results of the Western blot analysis of the inhibition of the omega-3 polyunsaturated fatty acids to the factors related to the infection of SARS-CoV-2 mediated by the activation of the trimethylamine-N-oxide.

FIG. 7 shows the results of the Western blot analysis of the promotion of the omega-3 polyunsaturated fatty acids to the factors related to the downstream signaling pathways mediated by the activation of the trimethylamine-N-oxide.

FIG. 8 shows the results of the omega-3 polyunsaturated fatty acids to the increase of the GSH/GSSG ratio mediated by the activation of the trimethylamine-N-oxide.

FIG. 9 shows the results of the Western blot analysis of the promotion of the omega-3 polyunsaturated fatty acids to the expression of γ-GCS mediated by the activation of the trimethylamine-N-oxide.

FIG. 10 shows the results of the omega-3 polyunsaturated fatty acids to the increase of the neovasculogenesis index mediated by the activation of the trimethylamine-N-oxide.

FIG. 11 shows the results of the Western blot analysis of the inhibition of the omega-3 polyunsaturated fatty acids to the factors related to the vascular fibrogenesis mediated by the activation of the trimethylamine-N-oxide.

### DETAILED DESCRIPTION

The present disclosure provides a novel use of omega-3 polyunsaturated fatty acids, especially a use of the omega-3 polyunsaturated fatty acids to regulate related responses mediated by an activation of a trimethylamine-N-oxide.

To illustrate the effects of the omega-3 polyunsaturated fatty acids in regulating a downstream signaling pathway mediated by the activation of the trimethylamine-N-oxide, the present disclosure will be further exemplified by the following specific embodiments. The person having ordinary skills in the art can fully utilize and implement the present disclosure without excessive interpretation. However, the readers should understand that the present disclosure should not be limited to these practical details thereof, that is, in some embodiments, these practical details are used to describe how to implement the materials and methods of the present disclosure and are not necessary.

Reagent formulations of control groups, comparison groups, and all of the embodiments of the present disclosure are listed in Table 1, Table 2, and Table 3, wherein the omega-3 polyunsaturated fatty acid is a docosahexaenoic acid (“DHA” hereafter) or an eicosapentaenoic acid (“EPA” hereafter).

TABLE 1

| | | omega-3 polyunsaturated | |
|---|---|---|---|
| | Trimethylamine-N- | fatty acid | |
| | oxide (μM) | DHA (μM) | EPA (μM) |
| Control group 1 | 0 | 0 | 0 |
| Comparison group 1 | 300 | 0 | 0 |
| Embodiment 1 | 300 | 25 | 0 |
| Embodiment 2 | 300 | 50 | 0 |
| Embodiment 3 | 300 | 125 | 0 |
| Embodiment 4 | 300 | 0 | 25 |
| Embodiment 5 | 300 | 0 | 50 |
| Embodiment 6 | 300 | 0 | 125 |

TABLE 2

| | | Trimethyl- | omega-3 |
|---|---|---|---|
| | | amine- | polyunsaturated |
| | SCF | N-oxide | fatty acid |
| | (ng/mL) | (μM) | DHA (μM) |
| Control group 2 | 0 | 0 | 0 |
| Comparison group 2 | 20 | 0 | 0 |
| Comparison group 3 | 20 | 300 | 0 |
| Embodiment 7 | 20 | 300 | 25 |
| Embodiment 8 | 20 | 300 | 50 |
| Embodiment 9 | 20 | 300 | 125 |

TABLE 3

| | | omega-3 polyunsaturated | |
|---|---|---|---|
| | Trimethylamine- | fatty acid | |
| | N-oxide (μM) | DHA (μM) | EPA (μM) |
| Control group 3 | 0 | 0 | 0 |
| Comparison group 4 | 50 | 0 | 0 |
| Embodiment 10 | 50 | 25 | 0 |
| Embodiment 11 | 50 | 50 | 0 |
| Embodiment 12 | 50 | 125 | 0 |
| Embodiment 13 | 50 | 0 | 25 |
| Embodiment 14 | 50 | 0 | 50 |
| Embodiment 15 | 50 | 0 | 125 |

In the present disclosure, DHA and EPA are purchased from Cayman Chemical Inc. (Ann Arbor, USA), and both DHA and EPA are in free fatty acid forms. The trimethylamine-N-oxide is purchased from Sigma-Aldrich (St. Louis, USA), and the trimethylamine-N-oxide is dissolved in dimethyl sulfoxide for subsequent experiments. Human recombinant stem cell factor (SCF) protein is purchased from R&D Systems (Minneapolis, USA).

I. Analysis of the Effects of the Omega-3 Polyunsaturated Fatty Acids in Inhibiting the Factors Related to the Downstream Signaling Pathway Mediated by the Activation of the Trimethylamine-N-Oxide

To analyze the situation of the omega-3 polyunsaturated fatty acids in inhibiting the downstream signaling pathway mediated by the activation of the trimethylamine-N-oxide, human endothelial progenitor cells (hEPCs) are used for the experiments. In detail, the human endothelial progenitor cells are cultured in the MCDB-131 medium supplemented with a 10% fetal bovine serum (FBS) and an EGM™-2 growth kit (Lonza, Inc., USA) and are administered separately with reagent formulations of the control group 1, the comparison group 1, and the embodiments 1 to 6 shown in Table 1. After the culture time of 24 hours has elapsed, proteins of the human endothelial progenitor cells of the control group 1, the comparison group 1, and the embodiments 1 to 6 are extracted and analyzed by the Western blot analysis to confirm the expressions of factors related to the NF-κB signaling pathway and the MAPK/p38 and JNK signaling pathway so as to evaluate the effects of the omega-3 polyunsaturated fatty acids on the factors related to the downstream signaling pathways mediated by the activation of the trimethylamine-N-oxide.

Further, the operation details of the Western blot analysis are well-known in the art and can be adjusted according to the experimental requirements, so the detailed steps thereof will not be described herein again.

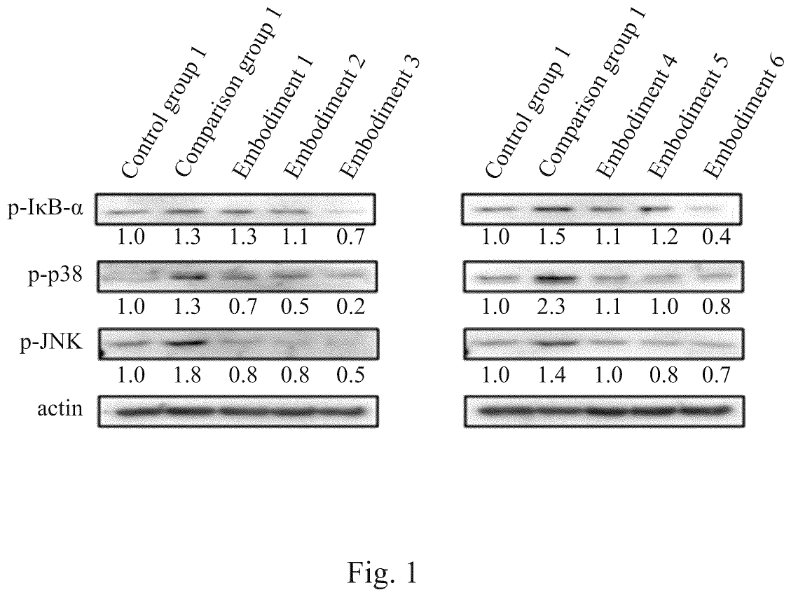

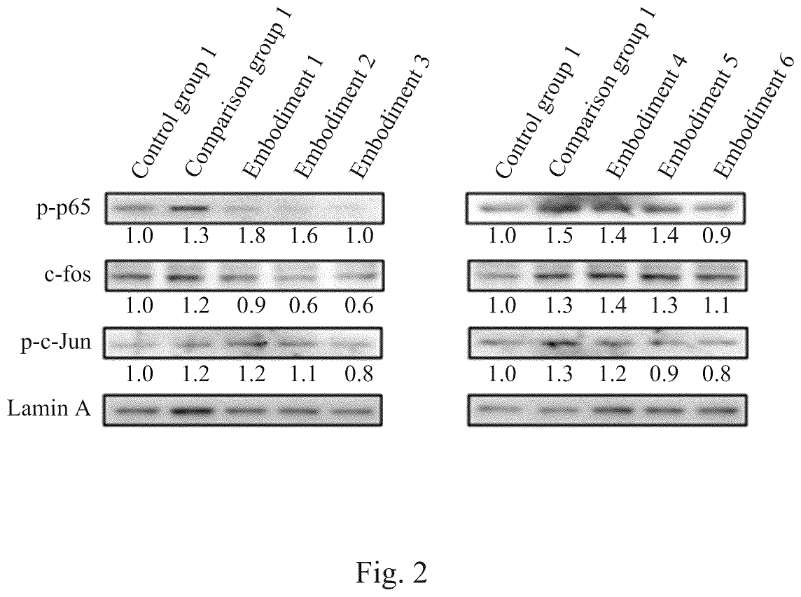

Reference is made to FIG. 1 and FIG. 2. FIG. 1 shows the results of the Western blot analysis of the inhibition of the omega-3 polyunsaturated fatty acids to the factors related to the downstream signaling pathways mediated by the activation of the trimethylamine-N-oxide. FIG. 2 shows the results of the Western blot analysis of the inhibition of the omega-3 polyunsaturated fatty acids to other factors related to the downstream signaling pathways mediated by the activation of the trimethylamine-N-oxide. In detail, p-IκB-α, p-p38, and p-JNK in FIG. 1 are cytoplasmic proteins involved in the NF-κB signaling pathway and the MAPK/p38 and JNK signaling pathway, whereas p-p65, c-fos, and p-c-Jun in FIG. 2 are nuclear proteins involved in the NF-κB signaling pathway and the MAPK/p38 and JNK signaling pathway.

As shown in FIG. 1 and FIG. 2, the expressions of p-IκB-α, p-p38, p-JNK, p-p65, c-fos, and p-c-Jun in the human endothelial progenitor cells of the embodiments 1 to 3 decrease along with the increase of the concentrations of DHA or EPA compared with the comparison group 1. The results show that the omega-3 polyunsaturated fatty acids such as DHA or EPA can effectively reduce the expressions of factors induced by the treatment with the trimethylamine-N-oxide and can reduce the expressions of p-IκB-α and p-p65 in the human endothelial progenitor cells so as to inactivate the NF-κB signaling pathway. Furthermore, DHA or EPA can significantly inhibit phosphorylation levels of p-p38 and p-JNK and the expressions of c-fos and p-c-Jun mediated by the activation of the trimethylamine-N-oxide so as to inactivate the MAPK/p38 and JNK signaling pathway.

According to the above description, the omega-3 polyunsaturated fatty acids can effectively inhibit the downstream signaling pathways mediated by the activation of the trimethylamine-N-oxide, so the method for regulating the downstream signaling pathway mediated by the activation of the trimethylamine-N-oxide of the present disclosure has the medical potential in relevant arts.

II. Analysis of the Effects of the Omega-3 Polyunsaturated Fatty Acids in Inhibiting Inflammatory Responses Induced by the Activation of the Trimethylamine-N-Oxide.

To analyze the effects of the omega-3 polyunsaturated fatty in inhibiting the inflammatory responses induced by the activation of the trimethylamine-N-oxide, human endothelial progenitor cells are cultured in the MCDB-131 medium supplemented with the 10% fetal bovine serum and the EGM™-2 growth kit and are administered separately with reagent formulations of the control group 1, the comparison group 1, and the embodiments 1 to 6 shown in Table 1. After the culture time of 24 hours has elapsed, the expressions of cytokines related to the inflammatory responses in the human endothelial progenitor cells are analyzed by enzyme-linked immunosorbent assay (ELISA) and quantitative PCR (qPCR) so as to evaluate the effects of the omega-3 polyunsaturated fatty acids in inhibiting the inflammatory responses mediated by the activation of the trimethylamine-N-oxide. Each experiment is repeated 3 times.

In detail, according to the previous studies, miRNA-221 (“miR-221” hereafter) plays an essential role in the physiological metabolism of the human endothelial progenitor cells. Treatment of the human endothelial progenitor cells with the anti-sense plasmid of miR-221 can effectively inhibit the expression of interleukin-6 (“IL-6” hereafter) induced by the activation of the trimethylamine-N-oxide. Therefore, the present disclosure uses the quantitative PCR and the enzyme-linked immunosorbent assay to analyze the expressions of miR-221 and IL-6 in the human endothelial progenitor cells of the control group 1, the comparison group 1, and the embodiments 1 to 6 after cell culture for 24 hours so as to evaluate the effects of the omega-3 polyunsaturated fatty acids in inhibiting the inflammatory responses induced by the activation of the trimethylamine-N-oxide.

Furthermore, the operation details of the quantitative PCR and the enzyme-linked immunosorbent assay are well-known in the art and can be adjusted according to the experimental requirements, so the detailed steps thereof will not be described herein again.

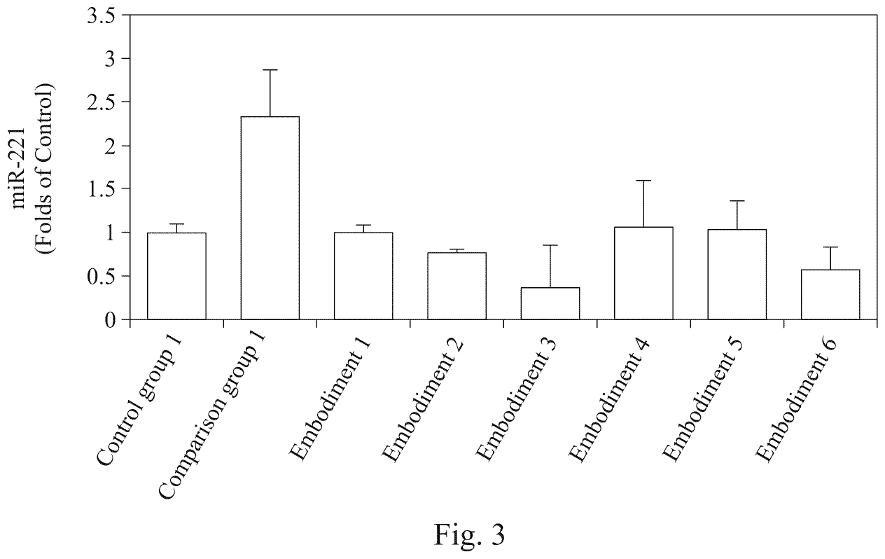

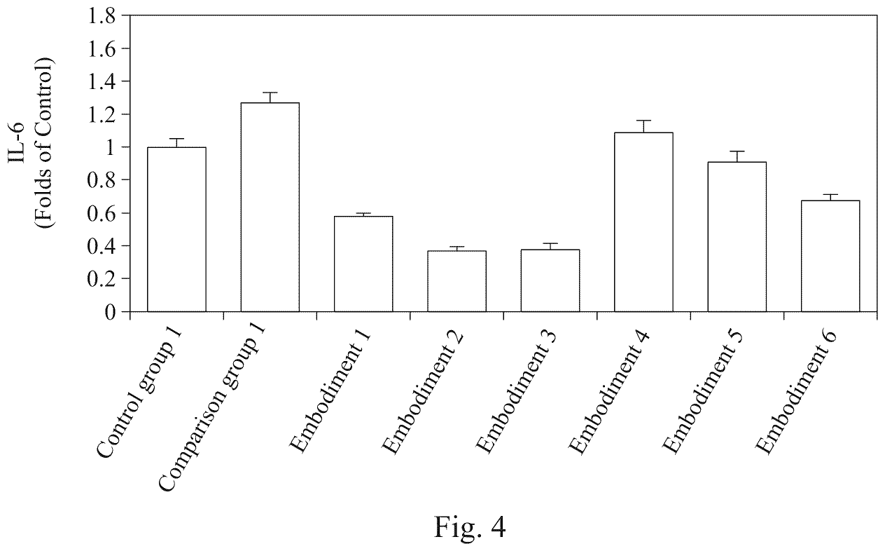

Reference is made to FIG. 3 and FIG. 4. FIG. 3 shows the results of the inhibition of the omega-3 polyunsaturated fatty acids to the expression of miR-221 mediated by the activation of the trimethylamine-N-oxide. FIG. 4 shows the results of the inhibition of the omega-3 polyunsaturated fatty acids to the expression of IL-6 mediated by the activation of the trimethylamine-N-oxide.

As shown in FIG. 3, the expression of miR-221 in the comparison group 1 is significantly higher than that in the control group 1, suggesting that the trimethylamine-N-oxide can notably promote the expression of miR-221 in the human endothelial progenitor cells. However, after the human endothelial progenitor cells are treated with DHA or EPA, the expression of miR-221 in the embodiments 1 to 6 is significantly lower than that in the comparison group 1 (p<0.05), suggesting that the omega-3 polyunsaturated fatty acids can effectively inhibit the expression of miR-221 induced by the activation of the trimethylamine-N-oxide. Further, compared with the comparison group 1, the effects of the embodiment 3 and the embodiment 6 on inhibiting the expression of miR-221 can reach 85% and 75% (p<0.05), respectively.

As shown in FIG. 4, after the human endothelial progenitor cells are treated with DHA or EPA, the expression of IL-6 in the embodiments 1 to 6 is significantly lower than that in the comparison group 1 (p<0.05), suggesting that the omega-3 polyunsaturated fatty acids can inhibit the expression of IL-6 induced by the activation of the trimethylamine-N-oxide by effectively inhibiting the expression of miR-221. Thus, the omega-3 polyunsaturated fatty acids can be used to inhibit the inflammatory response related to IL-6 activation, and the present disclosure has the potential application in relevant markets.

III. Analysis of the Effects of the Omega-3 Polyunsaturated Fatty Acids in Inhibiting SARS-CoV-2 Infection in Cells

The coronavirus disease 2019 (“COVID-19” hereafter) is an infectious disease caused by severe acute respiratory syndrome coronavirus 2 (“SARS-CoV-2” hereafter). The rapid spread of COVID-19 around the world poses a significant threat to human health and impacts the stability and development of human society.

In the process of SARS-CoV-2 infecting a cell, the spike protein of SARS-CoV-2 binds with the angiotensin-converting enzyme 2 (“ACE2” hereafter) on the cell surface and triggers the cleavage of the S1/S2 site of SARS-CoV-2 spike protein by the transmembrane serine protease 2 (“TMPRSS2” hereafter), allowing SARS-CoV-2 to fuse with the cell membrane and entry into the cell.

To analyze the inhibitory effects of the omega-3 polyunsaturated fatty acids on SARS-CoV-2 cell infection, 1×10⁵ human endothelial progenitor cells per well are co-cultured with 5 μL of SARS-CoV-2 pseudotyped Lentivirus (“SARS-CoV-2 plasmid” hereafter) in the MCDB-131 medium containing the EGM™-2 growth kit and the 10% fetal bovine serum in a 96-well plate. Then, the human endothelial progenitor cells are administered separately with reagent formulations of the control group 1, the comparison group 1, and the embodiments 1 to 6 shown in Table 1, and incubated in an incubator at 37° C. for 48 hours. After the culture time of 48 hours has elapsed, Bright-Glo™ Reagent is added to each well to detect the fluorescent signals of different groups. The proteins of the human endothelial progenitor cells are extracted and analyzed by the Western blot analysis to confirm the expressions of ACE2 and TMPRSS2. Each experiment is repeated 3 times.

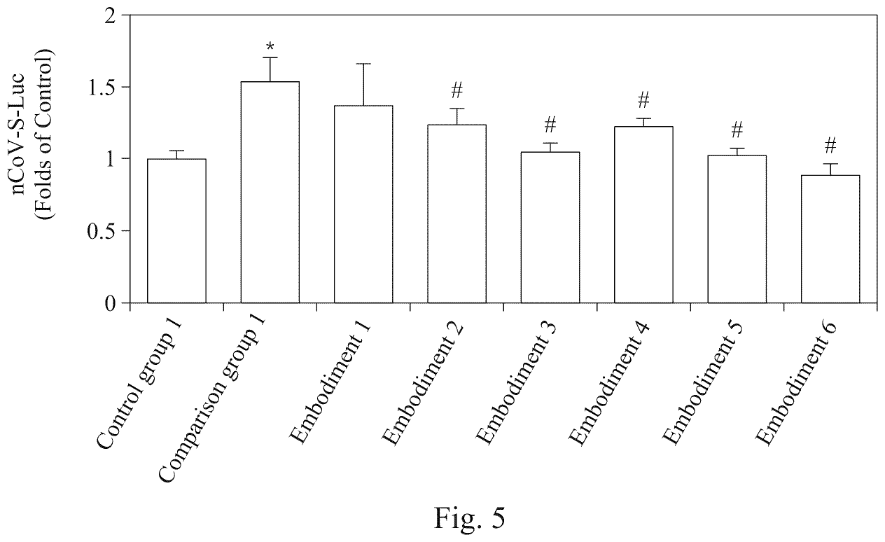

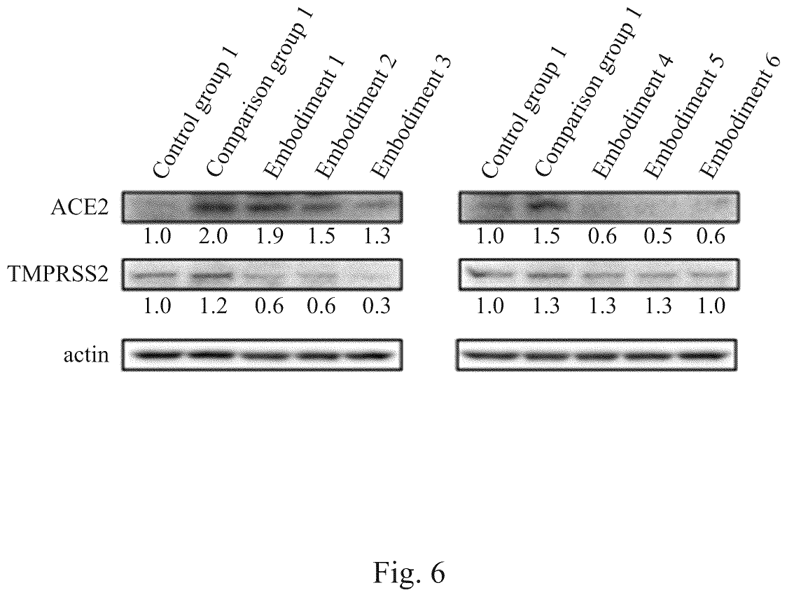

Reference is made to FIG. 5 and FIG. 6. FIG. 5 shows the results of the inhibition of the omega-3 polyunsaturated fatty acids to the trimethylamine-N-oxide activation-mediated entry of SARS-CoV-2 plasmid into human endothelial progenitor cells. FIG. 6 shows the results of the Western blot analysis of the inhibition of the omega-3 polyunsaturated fatty acids to the factors related to the infection of SARS-CoV-2 mediated by the activation of the trimethylamine-N-oxide. In FIG. 5, “nCov-S-Luc” indicated on the vertical axis represents the SARS-CoV-2 plasmid, “*” represents a statistical difference in comparison with the control group 1, and “#” represents a statistical difference in comparison with the comparison group 1. When there is a “*” mark or a “#” mark, it means that there is a significant difference (p<0.05) between the value of the comparison group 1 or the embodiment and the control group 1 or the comparison group 1.

As shown in FIG. 5, compared with the comparison group 1, the embodiments 1 to 3 containing DHA or the embodiments 4 to 6 containing EPA all significantly reduce the entry of SARS-CoV-2 plasmid into the human endothelial progenitor cells mediated by the activation of the trimethylamine-N-oxide. As shown in FIG. 6, the embodiments 1 to 6 can effectively reduce the expressions of ACE2 and TMPRSS2 induced by the activation of the trimethylamine-N-oxide, and the embodiments 1 to 3 containing DHA and the embodiments 4 to 6 containing EPA can effectively block the expressions of ACE2 and TMPRSS2. Thus, the method for regulating the downstream signaling pathway mediated by the activation of the trimethylamine-N-oxide of the present disclosure has the potential application in relevant markets.

IV. Analysis of the effects of the omega-3 polyunsaturated fatty acids in alleviating impairment of neovascularization induced by the activation of the trimethylamine-N-oxide.

Neovascularization is the process of de novo formation of blood vessels or formation of new blood vessels from pre-existing blood vessels. Neovascularization plays a key role in tissue regeneration and tissue repairment, wherein neovascularization is closely correlated to oxidative stress. When an oxidative stress level in a cell increases, it causes DNA damage and impairs neovascularization in the cell. The ratio of reduced glutathione (“GSH” hereafter) and oxidized glutathione (“GSSG” hereafter) has been noted as an oxidative stress index. Further, according to the previous studies, human stem cell factor (“SCF” hereafter) induces the activation of the Akt/endothelial nitric oxide synthase (“eNOS” hereafter) signaling pathway and the MAPK/ERK signaling pathway through the c-Kit receptor tyrosine kinase, thereby inducing neovascularization.

To analyze the effects of the omega-3 polyunsaturated fatty acids in alleviating the impairment of neovascularization induced by the activation of the trimethylamine-N-oxide, human endothelial progenitor cells are cultured in the MCDB-131 medium containing the EGM™-2 growth kit and the 10% fetal bovine serum in a Matrigel-coated 96-well plate. Then, the human endothelial progenitor cells are administered separately with reagent formulations of the control group 2, the comparison groups 2 to 3, and the embodiments 7 to 9 shown in Table 2, and incubated in an incubator at 37° C. for 8 hours.

After the culture time of 8 hours has elapsed, the human endothelial progenitor cells are fixed in glutaraldehyde/paraformaldehyde solution and treated with Calcein-AM solution so as to detect the fluorescent signals of different groups. The proteins of the human endothelial progenitor cells are extracted and analyzed by the Western blot analysis to confirm the expressions of factors related to the Akt/eNOS signaling pathway, the MAPK/ERK signaling pathway and the expression of gamma-glutamylcysteine synthetase (“γ-GCS” hereafter), wherein the γ-GCS participates in the synthesis of GSH. The GSH/GSSG ratios in the human endothelial progenitor cells are analyzed by using the liquid chromatography, the electrospray ionization, and the mass spectrometry. Each experiment is repeated 3 times.

The operation details of the liquid chromatography, the electrospray ionization, and the mass spectrometry are well-known in the art and can be adjusted according to the experimental requirements, so the detailed steps thereof will not be described herein again.

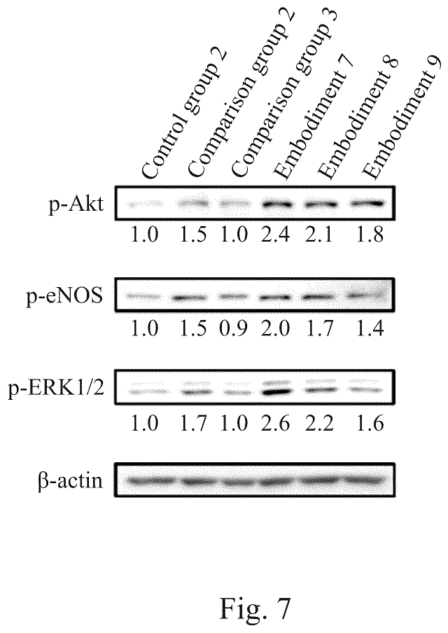

Reference is made to FIG. 7. FIG. 7 shows the results of the Western blot analysis of the promotion of the omega-3 polyunsaturated fatty acids to the factors related to the downstream signaling pathways mediated by the activation of the trimethylamine-N-oxide. In detail, p-Akt, p-eNOS, and p-ERK1/2 in FIG. 7 are cytoplasmic proteins involved in the Akt/eNOS signaling pathway and the MAPK/ERK signaling pathway.

As shown in FIG. 7, the expressions of p-Akt, p-eNOS, and p-ERK1/2 in the human endothelial progenitor cells of the comparison group 3 are lower than that in the comparison group 2, suggesting that the trimethylamine-N-oxide can inhibit the activation of the Akt/eNOS signaling pathway and the MAPK/ERK signaling pathway induced by SCF. However, after the human endothelial progenitor cells are treated with DHA, the expressions of p-Akt, p-eNOS, and p-ERK1/2 in the embodiments 7 to 9 are higher than that in the comparison group 3, suggesting that the omega-3 polyunsaturated fatty acids such as DHA can effectively promote the expressions of p-Akt, p-eNOS, and p-ERK1/2 in the human endothelial progenitor cells so as to promote the activation of the Akt/eNOS signaling pathway and the MAPK/ERK signaling pathway.

According to the above description, the omega-3 polyunsaturated fatty acids can effectively regulate the downstream signaling pathways mediated by the activation of the trimethylamine-N-oxide, so the method for regulating the downstream signaling pathway mediated by the activation of the trimethylamine-N-oxide of the present disclosure has the medical potential in relevant arts.

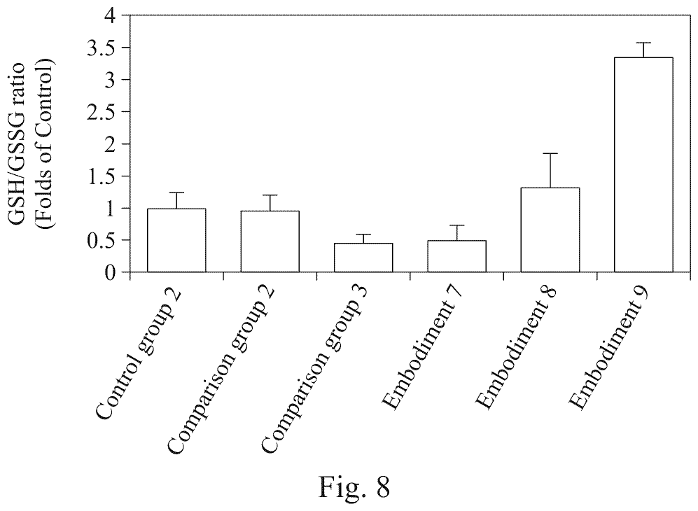

Reference is made to FIG. 8. FIG. 8 shows the results of the omega-3 polyunsaturated fatty acids to the increase of the GSH/GSSG ratio mediated by the activation of the trimethylamine-N-oxide. In detail, the GSH/GSSG ratio can reflect an oxidative stress level of a cell, and the GSH/GSSG ratio decreases with increased level of oxidative stress.

As shown in FIG. 8, the GSH/GSSG ratio in the human endothelial progenitor cells of the comparison group 3 is significantly lower than that of the comparison group 2 (p<0.05), suggesting that the trimethylamine-N-oxide can increase the oxidative stress level in the human endothelial progenitor cells. However, compared with the comparison group 3, after the human endothelial progenitor cells are treated with DHA, the GSH/GSSG ratio in the human endothelial progenitor cells of the embodiments 7 to 9 increases along with the increase of the concentrations of DHA. The results show that the omega-3 polyunsaturated fatty acids such as DHA can effectively increase the GSH/GSSG ratio mediated by the activation of the trimethylamine-N-oxide. Thus, the omega-3 polyunsaturated fatty acids can be used to reduce the oxidative stress level, and the present disclosure has the potential application in relevant markets.

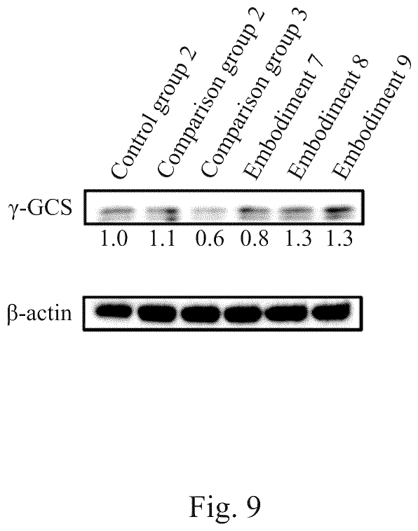

Reference is made to FIG. 9. FIG. 9 shows the results of the Western blot analysis of the promotion of the omega-3 polyunsaturated fatty acids to the expression of γ-GCS mediated by the activation of the trimethylamine-N-oxide. As shown in FIG. 9, the embodiments 7 to 9 can effectively promote the expression of γ-GCS mediated by the activation of the trimethylamine-N-oxide, suggesting that the omega-3 polyunsaturated fatty acids such as DHA can increase the GSH/GSSG ratio mediated by the activation of the trimethylamine-N-oxide by effectively promoting the expression of γ-GCS. Thus, the omega-3 polyunsaturated fatty acids can be used to promote the expression of γ-GCS, and the present disclosure has the potential application in relevant markets.

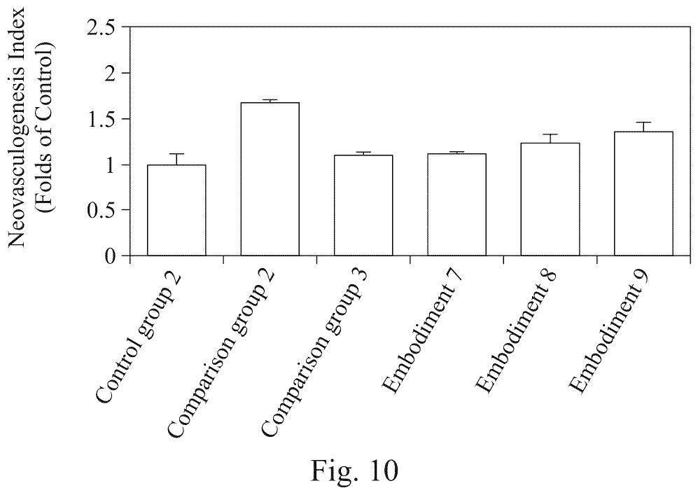

Reference is made to FIG. 10. FIG. 10 shows the results of the omega-3 polyunsaturated fatty acids to the increase of the neovasculogenesis index mediated by the activation of the trimethylamine-N-oxide. As shown in FIG. 10, the neovasculogenesis index of the comparison group 3 is significantly lower than that in the comparison group 2 (p<0.05), suggesting that the trimethylamine-N-oxide can inhibit the neovascularization mediated by SCF. However, after the human endothelial progenitor cells are treated with DHA, the neovasculogenesis indices of the embodiments 7 to 9 increase along with the increase of the concentrations of DHA compared with the comparison group 3. The results show that the omega-3 polyunsaturated fatty acids such as DHA can effectively alleviate the impairment of neovascularization induced by the activation of the trimethylamine-N-oxide. Thus, the method for regulating the downstream signaling pathway mediated by the activation of the trimethylamine-N-oxide of the present disclosure has the potential application in relevant markets.

V. Analysis of the Effects of the Omega-3 Polyunsaturated Fatty Acids in Inhibiting the Factors Related to Vascular Fibrogenesis Mediated by the Activation of the Trimethylamine-N-Oxide

Endothelial cells, which line the interior surface of blood vessels, play a crucial role in maintaining the structural and functional integrity of the vascular system and in regulating tissue repair. Evidence shows that the trimethylamine-N-oxide stimulates endothelial cells to transform into myofibroblasts (a process termed endothelial-mesenchymal transition), promotes the expressions of several fibrogenesis proteins, and induces extracellular matrix deposition, thereby promoting the vascular fibrogenesis. Vascular fibrogenesis plays an essential role in the progression of chronic diseases such as cardiovascular disease and systemic sclerosis, wherein the systemic sclerosis is a rare disease that involves the hardening and tightening of the skin, and there is no treatment method that can cure or stop the overproduction of fibrogenesis proteins.

To analyze the effects of the omega-3 polyunsaturated fatty acids in inhibiting the vascular fibrogenesis mediated by the activation of the trimethylamine-N-oxide, human umbilical vein endothelial cells (HUVECs) are used for the experiments. In detail, the human umbilical vein endothelial cells are cultured in the MCDB-131 medium supplemented with the 10% fetal bovine serum and the EGM™-2 growth kit and are administered separately with reagent formulations of the control group 3, the comparison group 4, and the embodiments 10 to 15 shown in Table 3. After the culture time of 24 hours has elapsed, proteins of the human umbilical vein endothelial cells of the control group 3, the comparison group 4, and the embodiments 10 to 15 are extracted and analyzed by the Western blot analysis to confirm the expressions of factors related to the vascular fibrogenesis so as to evaluate the effects of the omega-3 polyunsaturated fatty acids on the factors related to the vascular fibrogenesis mediated by the activation of the trimethylamine-N-oxide.

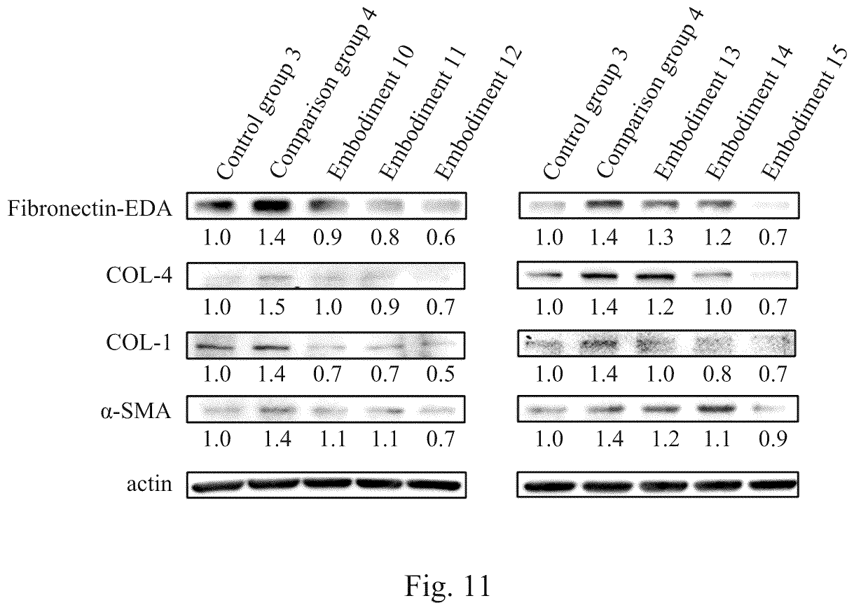

Reference is made to FIG. 11. FIG. 11 shows the results of the Western blot analysis of the inhibition of the omega-3 polyunsaturated fatty acids to the factors related to the vascular fibrogenesis mediated by the activation of the trimethylamine-N-oxide. In detail, Fibronectin-EDA, COL-4, COL-1, and α-SMA in FIG. 11 are fibrogenesis proteins involved in the vascular fibrogenesis and are myofibroblast markers for detecting the endothelial-mesenchymal transition.

As shown in FIG. 11, the expressions of Fibronectin-EDA, COL-4, COL-1, and α-SMA in the human umbilical vein endothelial cells of the embodiments 10 to 15 decrease along with the increase of the concentrations of DHA or EPA compared with the comparison group 4. The results show that the omega-3 polyunsaturated fatty acids such as DHA or EPA can effectively reduce the expressions of fibrogenesis proteins induced by the treatment with the trimethylamine-N-oxide, reduce the expressions of Fibronectin-EDA, COL-4, COL-1, and α-SMA in the human umbilical vein endothelial cells, and inhibit the endothelial-mesenchymal transition mediated by the activation of the trimethylamine-N-oxide so as to inhibit the vascular fibrogenesis.

According to the above description, the omega-3 polyunsaturated fatty acids can effectively inhibit the endothelial-mesenchymal transition mediated by the activation of the trimethylamine-N-oxide, and the omega-3 polyunsaturated fatty acids can be used to inhibit the vascular fibrogenesis induced by the endothelial-mesenchymal transition. Therefore, the method for regulating the downstream signaling pathway mediated by the activation of the trimethylamine-N-oxide of the present disclosure has the medical potential in relevant arts.

Further, in the present disclosure, the omega-3 polyunsaturated fatty acid can be administered to the subject as a solution, a suspension, an emulsion, a powder, a granule, a tablet, a pill, a syrup, a troche, a lozenge, a chewable gel, a magma, or a capsule as required. Furthermore, the omega-3 polyunsaturated fatty acid can be incorporated into pharmaceutical compositions. Such compositions can include one or more pharmaceutically acceptable carriers. The above-mentioned pharmaceutically acceptable carrier can include solvents, dispersion media, coatings, antibacterial and antifungal agents, isotonic and absorption delaying agents, and the like, compatible with pharmaceutical administration, but the present disclosure is not limited thereto.

Therefore, the present disclosure is based on the discovery of the relationship between the omega-3 polyunsaturated fatty acid and the downstream signaling pathway mediated by the activation of the trimethylamine-N-oxide, and thus the omega-3 polyunsaturated fatty acids can be used to regulate the responses caused by the downstream signaling pathways mediated by the activation of the trimethylamine-N-oxide. Further, the omega-3 polyunsaturated fatty acids can inhibit the inflammatory responses caused by the increase of IL-6 induced by the activation of the trimethylamine-N-oxide and can also reduce the entry of SARS-CoV-2 into cells mediated by the activation of the trimethylamine-N-oxide. Furthermore, the omega-3 polyunsaturated fatty acids can reduce the oxidative stress level caused by the activation of the trimethylamine-N-oxide and can alleviate the impairment of neovascularization induced by the activation of the trimethylamine-N-oxide. Moreover, the omega-3 polyunsaturated fatty acids can inhibit the vascular fibrogenesis induced by the promotion of endothelial-mesenchymal transition mediated by the activation of the trimethylamine-N-oxide. Therefore, the method for regulating the downstream signaling pathway mediated by the activation of the trimethylamine-N-oxide of the present disclosure has the potential application in relevant markets.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for regulating a downstream signaling pathway mediated by an activation of a trimethylamine-N-oxide, comprising:
administering an effective amount of an omega-3 polyunsaturated fatty acid to a subject in need thereof.

2. The method of claim 1, wherein the omega-3 polyunsaturated fatty acid is for inhibiting an inflammatory response.

3. The method of claim 2, wherein the omega-3 polyunsaturated fatty acid is for inhibiting the inflammatory response induced by an interleukin-6.

4. The method of claim 1, wherein the omega-3 polyunsaturated fatty acid is for inhibiting an infection of a SARS-CoV-2 to a cell.

5. The method of claim 4, wherein the omega-3 polyunsaturated fatty acid is for inhibiting an expression of an angiotensin-converting enzyme 2 and an expression of a transmembrane serine protease 2 in the cell.

6. The method of claim 4, wherein the cell is a human endothelial progenitor cell.

7. The method of claim 1, wherein the omega-3 polyunsaturated fatty acid is for reducing an oxidative stress level in a cell.

8. The method of claim 7, wherein the cell is a human endothelial progenitor cell.

9. The method of claim 1, wherein the omega-3 polyunsaturated fatty acid is for alleviating an impairment of neovascularization.

10. The method of claim 1, wherein the omega-3 polyunsaturated fatty acid is for inhibiting a vascular fibrogenesis.

11. The method of claim 10, wherein the omega-3 polyunsaturated fatty acid is for inhibiting the vascular fibrogenesis induced by an endothelial-mesenchymal transition in a cell.

12. The method of claim 11, wherein the cell is a human umbilical vein endothelial cell.

13. The method of claim 1, wherein the omega-3 polyunsaturated fatty acid is a docosahexaenoic acid or an eicosapentaenoic acid.

14. The method of claim 13, wherein the effective amount of the docosahexaenoic acid is 25 μM to 125 μM.

15. The method of claim 13, wherein the effective amount of the eicosapentaenoic acid is 25 μM to 125 μM.

16. The method of claim 1, wherein the omega-3 polyunsaturated fatty acid is administered to the subject by a solution, a suspension, an emulsion, a powder, a granule, a tablet, a pill, a syrup, a troche, a lozenge, a chewable gel, a magma, or a capsule.
